(19) United States
(12) Patent Application Publication
(10) Pub. No.: US 2022/0288266 A1
(43) Pub. Date: Sep. 15, 2022

(54) MASK WITH ULTRAVIOLET DISINFECTION OF INHALED AND EXHALED AIR

(71) Applicants: Alexander Gennadevich Mikhailov, Krasnoyarsk (RU); Olga Alexandrovna Kushnerova, Krasnoyarsk (RU); Svetlana Ramos, Virginia Beach, VA (US); Alexey Vladimirovich Kushnerov, Krasnoyarsk (RU)

(72) Inventors: Alexander Gennadevich Mikhailov, Krasnoyarsk (RU); Olga Alexandrovna Kushnerova, Krasnoyarsk (RU); Svetlana Ramos, Virginia Beach, VA (US); Alexey Vladimirovich Kushnerov, Krasnoyarsk (RU)

(21) Appl. No.: 17/331,699

(22) Filed: May 27, 2021

(30) Foreign Application Priority Data
Mar. 9, 2021 (RU) 2021106094

(51) Int. Cl. A61L 9/20; A62B 18/02; A62B 23/02; A62B 18/08; A42B 1/012

(52) U.S. Cl. CPC A61L 9/20; A62B 18/025; A62B 23/02; A62B 18/084; A62B 18/08; A42B 1/012; A61L 2209/12; A61L 2209/14; A61L 2209/15

(57) ABSTRACT

The mask with ultraviolet disinfection of inhaled and exhaled air in the form of a filtering-purification protective front surface contains a removable unit, the inner surface of which is covered with an ultraviolet radiation source with a pluggable independent power supply system. The filtering-purification unit has rows of meshes composed of light guides of ultraviolet radiation spectrum with tapped radiation in each cell of the mesh, and a source is brought to the meshes. The mask itself can be made of a mesh composed of light guides. The mask in the form of a shield in front of the face is represented by a mesh composed of light guides in front of the face or along the entire perimeter. The mask along the bottom part of the headband, above the face, is provided with a LED strip of ultraviolet radiation spectrum, radiation being directed downward, the sector of directed radiation being adjustable. The headgear can serve as a headband. Also, the LED radiation strip is attached to the eyeglasses along the perimeter of the spectacle frame.

The invention relates to a filtering-purification face mask for purifying and disinfecting inhaled and exhaled air. Masks are usually used to protect the respiratory tract of a person for at least the following common reasons: to prevent the ingress of suspended particulate matter into the respiratory system; to protect against the ingress of polluting substances and pathogenic microorganisms and to protect other people or objects from exhaled microorganisms. The basis of the mask is a filtering-purification part, which includes a filtering unit that performs the function of purifying from mechanical impurities, or of disinfecting inhaled and exhaled air. To date, a variety of masks and respirators have been developed, both for each individual purpose and for complex action. Most devices can be subsumed under a category of filtering face masks, which, in contrast to conventional respirators, use synthetic fabric bases in is combination with filtering cartridges to be attached thereto (see, for example, U.S. Pat. No. RE39493 (Yuschak et al.)), or with filter inserts (see, for example, U.S. Pat. No. 4,790,306 (Braun)), where the mask part requires the filtering cartridge to be occasionally replaced. There are respirators in the form of a molded filtering face mask of a cup-shaped configuration. The molded respirators usually have a permanent shape when in use and when stored. Examples of molded respirators in the form of filtering face mask include U.S. Pat. No. 7,131,442 (Kronzer et al.), U.S. Pat. No. 6,923,182, No. 6,041,782 (Angajivand et al), U.S. Pat. No. 4,807,619 (Dayrud and others), and U.S. Pat. No. 4,536,440 (Berg).

A closer analogue is the principle of disinfection of inhaled air exposed to ultraviolet irradiation (see, for example, patent (RU No. 2440147, U.S. Pat. No. 7,175,814, U.S. No. 6,730,265), usually of a stationary type, which disinfects the air by ultraviolet radiation, including by way of a forced passage of air through the radiation zone). Further, a safe range of the ultraviolet radiation spectrum for human skin is known. (https://vc.ru/tribuna/156964). The safe wavelength range is in the range of 220-230 nm. This range is safe for humans, with the air being disinfected along with the standard radiation with a wavelength of 254-280 nm. That is, disinfection is carried out by such ultraviolet radiation without posing a risk for humans. LED sources of the ultraviolet range, including ribbon-type ones, are now quite widespread and available. However, they are not intended for mobile use and safe individual short and long term use.

In order to achieve the purpose of providing a light mask (respirator) ensuring effective and high-quality disinfection of inhaled and exhaled air, as well as easy use and long-duration protection, the mask with an opening for inserting a filtering-purification unit for purifying inhaled air as well as a headband with elements for attaching the respirator to the user's head is distinctive in that the filtering-purification unit is designed to be removable and hermetically fastenable in the mask opening, and the inner surface of the replaceable unit is covered with an ultraviolet radiation source with a pluggable independent power supply system. Ultraviolet radiation in the unit is isolated from the external environment. In this case, any effective range of the UV spectrum can be applied. The removable unit designed in the form of a cylinder or rectangular box has no internal obstacles for free movement of air during inhalation and exhalation. The very shape of the air duct can be made in the form of a labyrinth or a spiral to increase the time of passage of inhaled and exhaled air in order to provide for an increased level of disinfection.

The mask is distinctive in that the filtering-purification unit has rows of meshes composed of light guides with tapped radiation in each cell of the mesh, and a source of ultraviolet radiation is brought to the light guides. The mesh with ultraviolet radiation provides for close contact with the air passing therethrough, which provides for quick and high-quality disinfection of the inhaled and exhaled air volumes. Several rows of mesh can be mounted in a replaceable unit for purifying inhaled and exhaled air. The intensity and consumed radiation power can be low, which allows for long-term use.

The mask is distinctive in that the entire surface of the mask is designed as a mesh composed of light guides, which is fastened to the frame.

The mask is distinctive in that a mesh composed of light guides is fastened to the rim of the headgear. The mask in the form of an ultraviolet face shield protects the entire facial region, which is its advantage. Radiation is controlled according to the directionality of ultraviolet radiation to the interior of the cells for the safety of others, or a safe spectrum of radiation is used.

The mask is distinctive in that a tape of LED radiation is laid along the lower part of the rim of the headgear. Such an ultraviolet face shield can be represented by a headgear, the face area of which or the entire perimeter of the headgear is provided with a LED strip of ultraviolet radiation spectrum.

The mask is distinctive in that the sources of LED ultraviolet radiation on the rim of the headgear have adjustable sectoral directed radiation. Radiation is directed downward by sectors, the angle of the radiation sector being adjustable. This version of the ultraviolet shield can be designed to be removable, which makes it suitable for attaching to other headgear, such as cap, hat, baseball cap, bandana, etc.

The mask according to claims 5, 6, is distinctive in that the LED strip of ultraviolet radiation is attached to the eyeglasses along the perimeter of the spectacle frame.

### EXAMPLE EMBODIMENT

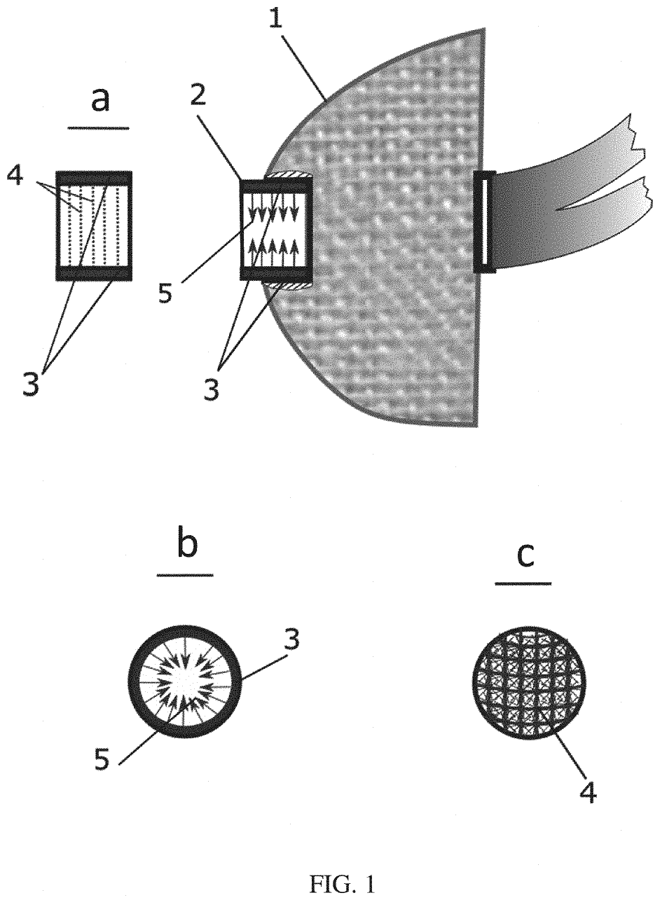

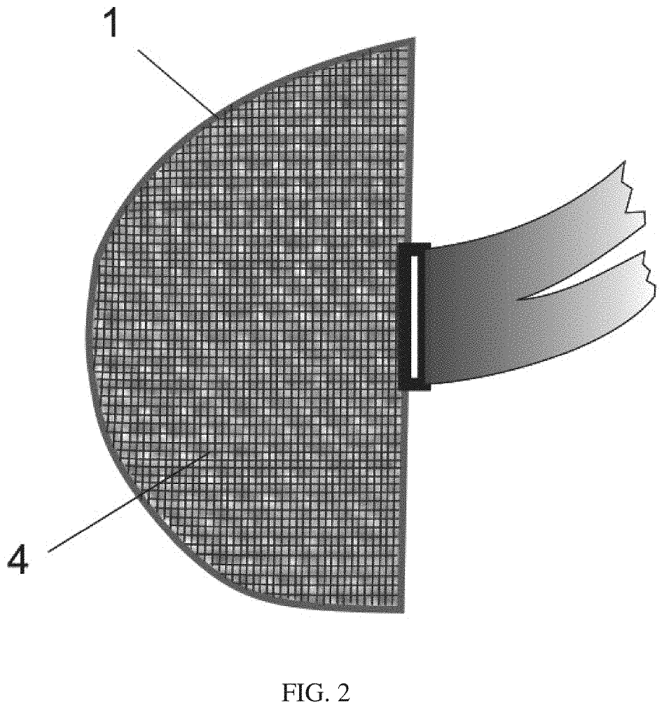

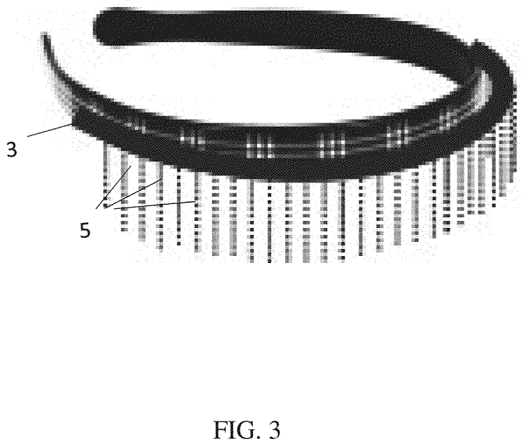

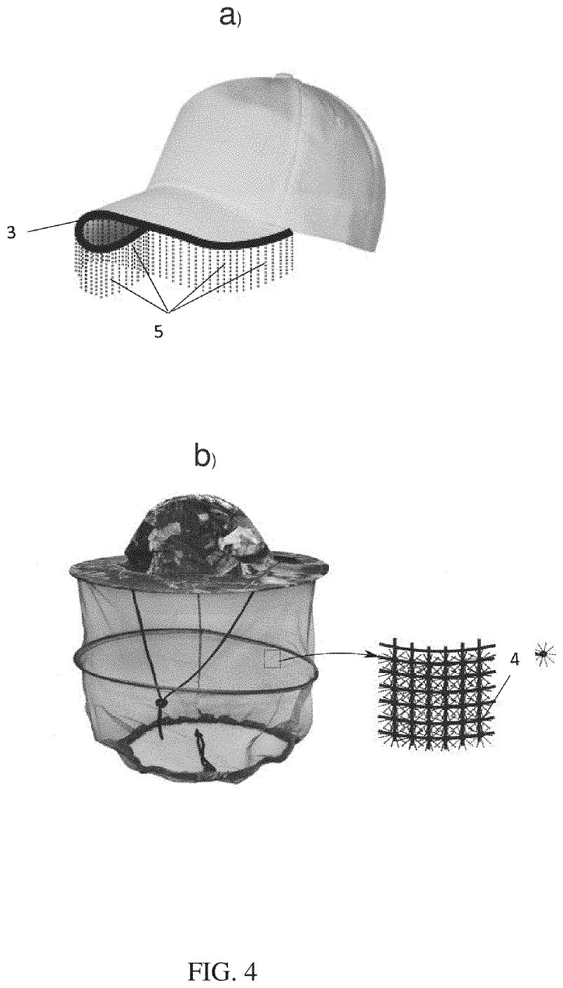

FIG. 1 shows a mask with replaceable disinfecting units, FIG. 1a shows a removable unit with a mesh composed of light guides with side glow; FIG. 1b shows a removable purification unit with a source along the inner perimeter, front view; FIG. 1c shows a removable unit with a mesh composed of light guides, front view; FIG. 2 shows a mask composed of light guides, FIG. 3 shows a headband rim with an ultraviolet “curtain”, FIG. 4 shows versions of the ultraviolet “curtain” (shield) on headgears, including those in FIG. 4a—on a baseball cap, in FIG. 4b—on a mosquito net hat, where: 1—a mask, 2—a removable purification unit, 3—an ultraviolet radiation source, 4—a mesh composed of light guides, 5—direction of ultraviolet radiation.

The respirator is operated as follows.

A removable purification unit 2 is inserted into mask 1. In the removable purification unit 2, an ultraviolet radiation source 3 is disposed on the inner surface, the radiation of this source being directed from the periphery of the removable purification unit 2 to the center. When the mask 1 is attached to the face, the inhaled and exhaled air passes through the removable purification unit 2. The air that passes through the removable purification unit 2 is exposed to wave irradiation of ultraviolet spectrum. When inhaled, the disinfected air enters the human respiratory system. When exhaled, the air from the human respiratory system passes through the removable purification unit 2 and is also exposed to ultraviolet irradiation, which disinfects the air to be exhaled into the environment.

In the system purification unit 2, a mesh 4 composed of light guides in one or more layers perpendicularly to the air movement direction can be used instead of the ultraviolet radiation source 3. In this case, the inhaled and exhaled air thanks to passing through the mesh has a closer contact with the ultraviolet radiation source, because there is a side radiation outlet inside the cell in each cell of the mesh in the mesh 4 composed of light guides. Disinfection in this case may have a higher level.

An ultraviolet “curtain” attached to the head can be used for disinfection of inhaled and exhaled air (FIG. 3, 4). Structurally, the ultraviolet “curtain” is a rim or the like attached to the head. Ultraviolet radiation from the source disposed along the perimeter propagates directionally downwards from the rim. Also, a mesh composed of light guides with ultraviolet radiation with side glow in each cell of the mesh directed to the interior of the cell can be used.

It is also possible to dispose the LED strip along the perimeter on the rim along the entire perimeter or only on the front part with the radiation being directed in a narrow sector.

The inhaled and exhaled air passes through the UV treatment area and undergoes disinfection. The version with a LED strip increases the mobility of application of such disinfection. The LED strip can be used on almost any headgear: hat, flat cap, baseball cap, mosquito net hat etc. The use of LED strip of an ultraviolet radiation spectrum that is safe for humans decreases limitations of field of use.

1. A mask with ultraviolet disinfection of inhaled and exhaled air, which has an opening for inserting a filtering-purification unit for purifying inhaled air as well as a headband with elements for attaching the respirator to the user's head, characterized in that the filtering-purification unit is designed to be removable and hermetically fastenable in the mask opening, and the inner surface of the replaceable unit is covered with an ultraviolet radiation source with a pluggable independent power supply system.

2. The mask according to claim 1, wherein the filtering-purification unit has rows of meshes composed of light guides with tapped radiation in each cell of the mesh, and a source of ultraviolet radiation is brought to the light guides.

3. The mask according to claim 2, wherein the entire surface of the mask is designed as a mesh composed of light guides, which is fastened to the frame.

4. The mask according to claim 3, wherein a mesh composed of light guides is fastened to the rim of the headgear.

5. The mask according to claim 4, wherein a tape of LED radiation is laid along the lower part of the rim of the headgear.

6. The mask according to claim 5, wherein the sources of LED ultraviolet radiation on the rim of the headgear have adjustable sectoral directed radiation.

7. The mask according to claim 5, wherein the LED strip of ultraviolet radiation is attached to the eyeglasses along the perimeter of the spectacle frame.
